(19) United States
(12) Patent Application Publication
(10) Pub. No.: US 2014/0322260 A1
(43) Pub. Date: Oct. 30, 2014

(54) MALIGNANT TUMOR TREATMENT AGENT AND FOOD AND DRINK INCLUDING SAME

(71) Applicant: Michio TANI, Meguro-ku, Tokyo (JP)

(72) Inventor: Michio Tani, Meguro-ku (JP)

(21) Appl. No.: 14/357,351

(22) PCT Filed: Nov. 12, 2012
(86) PCT No.: PCT/JP2012/079877

§ 371 Date: May 9, 2014

(30) Foreign Application Priority Data
Nov. 10, 2011 (JP) 2011-246976

(51) Int. Cl. A61K 36/8994; A61K 36/07; A61K 36/185; A61K 36/74; A61K 36/899; A23L 2/52; A61K 36/28; A61K 36/534; A61K 9/14; A61K 9/00; A23L 1/29; A61K 36/062; A61K 36/23

(52) U.S. Cl. CPC A61K 36/8994; A61K 36/062; A61K 36/07; A61K 36/185; A61K 36/74; A61K 36/899; A61K 36/23; A61K 36/28; A61K 36/534; A61K 9/14; A61K 9/0095; A23L 1/296; A23L 2/52
USPC 424/195.15

(57) ABSTRACT

It is an object of the present invention to provide a malignant tumor treatment agent that causes no serious side effects as seen in medicine and is highly safe even in long-term daily use because it is made of food material(s), while being superior to the capabilities of the conventional malignant tumor treatment agents, and food and drink including the same. A malignant tumor treatment agent comprising, as an active ingredient, a mixture obtained by mixing together MUGI KOJI (koji malt), SHIITAKE mushroom (Lentinula edodes), coix seed, cacao, nutmeg, coffee, gansun (bamboo shoots), parsley, stevia, and mint. The malignant tumor treatment agent of the present invention and the food and drink including the same show significant effects on malignant tumor treatment. Furthermore, because the food materials are used as raw material, the treatment agent and the food and drink cause no serious side effects as seen in radiation therapy and chemotherapy and are highly safe. In addition, the food and drink of the present invention can be used to continuously treatment malignant tumor in daily life.

### TECHNICAL FIELD

The present invention relates to a malignant tumor (such as cancer and leukemia) treatment agent, and to food and drink including the same. In particular, the invention relates to a malignant tumor treatment agent including, as an active ingredient, a mixture obtained by mixing MUGI KOJI (koji malt), SHIITAKE mushroom (Lentinula edodes), coix seed, cacao, nutmeg, coffee, gansun (bamboo shoots), parsley, stevia, and mint, and to food and drink including the same.

### BACKGROUND ART

Cancer and AIDS are obstinate diseases threatening people all over the world. Particularly, cancer accounts for approximately 30% of mortality in our country and is a serious disease difficult to treat even with the three major treatment modalities (surgery, radiation therapy, and anticancer chemotherapy) in modem medicine. And until now, in advanced cancer cases, it has been thought impossible to effect a preservative cure or obtain treatment effect up to presumed cure without causing severe side effects, except for a very few exceptional cases of naturally cured cancer. On the other hand, even if anticancer drugs are developed that can show a certain level of treatment effect in vitro or in laboratory animal studies, it is, in fact, difficult to cure cancer without side effects when practically used in humans. Accordingly, there is a desperate desire for development of therapeutic agents against malignant tumors (such as cancer and leukemia), which can show sufficient treatment effect and achieve cure at a significant constant rate without any side effects in practical medicine. This is because, under the circumstances in which so many lives in the world have been lost due to cancer, it is one of the most important and final goals of medicine to protect humans from cancer and cure the disease.

Under such situations, drugs with fewer side effects have been under development. Those drugs have different approaches from surgery, radiation therapy, and chemotherapy, although satisfactory results have not yet been obtained so far.

For example, among conventional anticancer drugs safe and having no side effect, there is an anticancer agent including, as an active ingredient, a physiologically active substance prepared by extracting at least one mushroom selected from BUNA SHIMEJI mushroom (brown beech mushroom) (Hypsizygus marmoreus), SHIITAKE mushroom (Lentinula edodes), MAITAKE mushroom (Hen of the Woods) (Grifola frondosa), HATAKE SHIMEJI mushroom (Lyophyllum decastes), and ENOKITAKE mushroom (Flammulina velutipes) by using water, a hydrophilic solvent, or a mixture solvent thereof. The anticancer agent is known to be able to enhance the anticancer effects of chemotherapeutic agents while maintaining high safety, and also to prevent cancer through its daily use (for example, see Patent Literature 1). However, the anticancer agent is still a drug for preventing cancer and enhancing the anticancer effects of chemotherapeutic agents, and there is no description about anticancer effect of the agent itself.

Additionally, there is known a cancer-inhibiting drug for prevention of diabetes and hepatitis. The cancer-inhibiting drug is characterized in that REISHI (bracket fungus) (Ganoderma lucidum) and a mushroom such as AGARICUS mushroom (Agaricus subrufescens), YAMABUSHITAKE mushroom (lion's mane mushroom) (Hericium erinaceum), ENOKITAKE mushroom (Flammulina velutipes), SHIITAKE mushroom (Lentinula edodes), MAITAKE mushroom (Hen of the Woods) (Grifola frondosa), SHIMEJI mushroom (Lyophyllum shimeji), or KIKURAGE mushroom (cloud ear mushroom) (Auricularia auricula-judae) are processed with strong alkali to be deacetylated and processed with strong acid to be deoxidized; the obtained solutions each are filtered off and mixed together for neutralization; then, calcium chloride is fed to be added into the deacetylated solution to filter off precipitated calcium beta-glucan; the obtained solution is condensed to recover a polysaccharide, which is absorbed in a powder of rice bran, soy beans, bean-curd lees (soy pulp), or Tianqi (panax pseudoginseng) (Panax notoginseng) to produce a dry powder; then, ascorbic acid, citric acid, and the like are processed to be mixed into the powder to obtain powdered ascorbate-citrate calcium beta-glucan; and the powdered calcium beta-glucan is mixed with stevia, luo han guo (Siraitia grosvenorii), chitin chitosan and amino acid, spore of REISHI (bracket fungus) (Ganoderma lucidum), Canarium album (Burseraceae), green tea powder, nonchlorella, vanillin, and/or pyrethroid to produce the drug (for example, see Patent Literature 2). However, the cancer-inhibiting drug has been prepared by processing SHIITAKE mushroom (Lentinula edodes) or MAITAKE mushroom (Hen of the Woods) (Grifola frondosa) with strong alkali or the like, so that it is different from the malignant tumor treatment agent of the present invention.

Additionally, as an agent using coffee that is a constituent component of the present invention, there is also known an adjuvant including, as an active ingredient, an astaxanthin-containing yeast or a substance that is a processed product of the yeast and includes a cell wall or a constituent component thereof and astaxanthin, in which the adjuvant further includes buckwheat (Fagopyrum esculentum), gambir (Uncarina gambir), coffee, cha (Camellia sinensis), hawthorn (Crataegus cuneata), turmeric or garlic, or antioxidative components contained therein, as an antioxidative substance suppressing oxidation of astaxanthin (for example, see Patent Literature 3).

However, the active ingredient of the adjuvant is an astaxanthin-containing yeast or a processed product of the yeast, while coffee is included merely as an antioxidative component. In addition, the invention has described only an immune reinforcement effect but not anything about cancer inhibiting function obtained thereby.

Meanwhile, there is a problem that there is no any malignant tumor treatment agent that does not cause serious side effects or the like as seen in medicine and is highly safe even in long-term daily use because it is made of food material(s), and also there is no food and drink including the treatment agent. In Patent Literature 4, the applicants have already proposed “a malignant tumor treatment agent including, as an active ingredient, a mixture obtained by mixing together SHIITAKE mushroom (Lentinula edodes), cacao, nutmeg, coffee, and stevia, in which the mixture is in a dry powder state and a dry powder prepared by freeze-drying an extract obtained by hot water extraction of a mixture obtained by mixing together SHIITAKE mushroom (Lentinula edodes), cacao, nutmeg, coffee, and stevia in a weight ratio in respective dry states of 3:3:2:2:1”.

### CITATION LIST

### Patent Literature

PTL 1: Japanese Laid-Open Patent Publication No. 2003-231644 A

PTL 2: Japanese Laid-Open Patent Publication No. 2004-010605 A

PTL 3: Japanese Laid-Open Patent Publication No. 2002-080351 A

PTL 4: Japanese Patent No. 4681363 B

### SUMMARY OF INVENTION

### Technical Problem

In view of the circumstance as described above, it is an object of the present invention to provide a malignant tumor treatment agent that causes no serious side effects as seen in medicine and is highly safe even in long-term daily use because it is made of food material(s), while being superior to the capabilities of the conventional malignant tumor treatment agents, and food and drink including the same.

### Solution to Problem

The above object is achieved by a malignant tumor treatment agent including, as an active ingredient, a mixture obtained by mixing together MUGI KOJI (koji malt), SHIITAKE mushroom (Lentinula edodes), coix seed, cacao, nutmeg, coffee, gansun (bamboo shoots), parsley, stevia, and mint.

In addition, an extract obtained by hot water extraction of the mixture may be included as the active ingredient; the mixture may be in a dry powder state; and a weight ratio in respective dry states of the MUGI KOJI (koji malt), SHIITAKE mushroom (Lentinula edodes), coix seed, cacao, nutmeg, coffee, gansun (bamboo shoots), parsley, stevia, and mint may be 3.2:3.0:2.7:2.5:1.9:1.8:1.0:0.9:0.8:0.7.

Additionally, the above object is achieved by food and drink including the above-described malignant tumor treatment agent.

### Advantages Effects of Invention

The malignant tumor treatment agent of the present invention and the food and drink including the same show significant effects on malignant tumor treatment through continuous use. Furthermore, because the food materials are used as raw material, the treatment agent and the food and drink cause no serious side effects as seen in radiation therapy and chemotherapy and are highly safe. In addition, the food and drink of the present invention can be used to continuously treatment malignant tumor in daily life.

### BRIEF DESCRIPTION OF DRAWINGS

FIG. 1 shows a table of the progress of cases in the administration of a malignant tumor treatment agent according to the present invention.

FIG. 2 is a transitive graph of a tumor marker CA 125 before and after administration of the malignant tumor treatment agent according to the present invention.

FIG. 3 is an X-ray photograph of the lungs showing the occurrence of malignant pleural effusion before the administration of the malignant tumor treatment agent according to the present invention.

FIG. 4 is an X-ray photograph of the lungs after the administration of the malignant tumor treatment agent according to the present invention.

FIG. 5 is an X-ray photograph of vertebral disappearance due to cancer metastasis before the administration of the malignant tumor treatment agent according to the present invention.

FIG. 6 is an X-ray photograph of vertebrae after the administration of the malignant tumor treatment agent according to the present invention.

### DESCRIPTION OF EMBODIMENTS

Hereinafter, a detailed description will be given of embodiments of the present invention.

It is known that even a perfectly healthy person, usually, has at least 500 to 1000 or more cancerous cells in the body. However, the cancerous cells do not develop into carcinoma, because inherent immune functions immediately find cancerous cells as abnormal ones and destroy them before they can become a tumor. Cancer-causing factors seem complicated. Briefly, they are based on two abnormalities: some cause (severe stress or negative effects of eating habits and environments) reduces the immune functions or causes an abnormally large number of cancer cells to be created. Then, from the viewpoint of Oriental medicine, to recover both abnormalities, there is obviously a need for recovery and reinforcement of the immune functions from three aspects: 1) quality improvement of the immune functions; 2) reinforcement of the immune functions; and 3) collaboration of the immune functions and improvement of environment thereof. In other words, to recover and reinforce the immune functions, a malignant tumor treatment agent prepared as in the present invention is effective. (In addition, to enhance treatment effect, it is empirically known that no alcohol and no smoking, together with vegetable diet, are more effective.)

A malignant tumor treatment agent according to the present invention includes, as an active ingredient, a mixture obtained by mixing together MUGI KOJI (koji malt), SHIITAKE mushroom (Lentinula edodes), coix seed, cacao, nutmeg, coffee, gansun (bamboo shoots), parsley, stevia, and mint The MUGI KOJI (koji malt) may be made by using organic wheat produced by a KOJI mold having “EKI (defensive qi (defensive spirit))” most suitable to enhance the immune functions from the viewpoint of the Oriental medicine, as a KOJI mold. The coix seed to be used is desirably those obtained from wild adlay with the husks removed. Gansun (bamboo shoots) may be made by cutting MADAKE bamboo (Phyllostachys bambusoides) shoots into narrow slits, steaming and salting them, and then fermenting with lactic acid, followed by drying them in the sun. As the cacao, particularly, cacao nib may be used, and as for the coffee, it is desirable to use beans dried without roasting.

In addition, in the malignant tumor treatment agent of the present invention, when a weight ratio in respective dry states of MUGI KOJI (koji malt), SHIITAKE mushroom (Lentinula edodes), coix seed, cacao, nutmeg, coffee, gansun (bamboo shoots), parsley, stevia, and mint is 3.7 to 2.7:3.5 to 2.5:3.2 to 2.2:3.0 to 2.0:2.4 to 1.4:2.3 to 1.3:1.5 to 0.5:1.4 to 0.4:1.3 to 0.3:1.2 to 0.2, preferable treatment effect is obtainable, and when the weight ratio thereof is 3.2:3.0:2.7:2.5:1.9:1.8:1.0:0.9:0.8:0.7, particularly preferable treatment effect is obtainable.

When taking the malignant tumor treatment agent of the present invention, the mixture may be cut into small pieces to be taken as it is, but it is preferable to take an extract obtained from hot-water extraction of the mixture. Additionally, the mixture may be in a dry powder state, and it is preferable to take a product prepared by figure processing of the dry powder or a hot-water extract.

Upon hot water extraction, the mixture or a dry product of the mixture as it is may be extracted in hot water or may be pulverized into smaller pieces to be extracted from a practical aspect. In addition, a weight ratio between hot water used as extraction solvent and the dry product is not specifically restricted, but preferably, hot water is in an amount of 10 to 50 weight times the weight of the dry product and, particularly in terms of extraction operation and efficiency, is in an amount of 20 to 40 weight times that of the dry product. Regarding extraction temperature, high temperature is efficient, and particularly a temperature of 70 to 95 degree Celsius is preferable. Extraction time is preferably 30 minutes or more and 60 minutes or less under normal pressure. In addition, extraction may be performed either under applied pressure or under normal pressure. Particularly preferable conditions are as follows: under normal pressure, the extraction temperature ranges from 75 to 90 degree Celsius, and the extraction time ranges from 30 to 60 minutes.

Furthermore, a product obtained by pulverizing the hot water extract by spray drying, freeze drying, or the like may be taken. Additionally, an excipient may be added to the pulverized product, and then the resulting product may be compressed into tablet form or processed into granular form to obtain a dosage form of tablets or granules.

Dosage of the malignant tumor treatment agent of the present invention can vary depending on the condition of the disease, the age of the patient, and the like, but usually, a preferable weight of the dry product per day is 10 to 30 g, and when taking the agent as a hot water extract, it is appropriate to take a product obtained by extracting the above amount of the dry product in 200 to 500 g of hot water. When taking it as an extract powder, granules, a tablet, or the like prepared by spray drying or freeze drying a hot water extract solution, 3.0 to 6.0 g is appropriate as a weight of the powdered product.

In addition, food and drink according to the present invention can be provided by mixing the malignant tumor treatment agent of the present invention into general foods including soups, various kinds of drinks (such as juice, alcohol, and mineral water), confectionary (such as chewing gum, candies, chocolate, snacks, and jelly), and noodles (such as SOBA noodles (buckwheat noodles), UDON noodles (wheat noodles), and RAMEN noodles (Chinese noodles)), health foods, and nutritional supplements (such as nutritional drinks). Thereby, the malignant tumor treatment agent can be taken without reluctance in daily life.

In addition, concentration of the malignant tumor treatment agent contained in the drink and food of the present invention can be appropriately changed depending on the kind of the drink and food thereof. Usually, when mixing the dry powder, it may be mixed such that an intake per day is 10 to 50 g, and preferably 20 to 30 g. An extract powder obtained by spray drying or freeze drying the hot water extract solution may be mixed such that an intake per day is 2.0 to 12.0 g, and preferably 3.0 to 6.0 g. The above concentrations are one example and can be appropriately changed according to various situations.

### EXAMPLES

Hereinafter, the present invention will be described in more detail using Examples, but is not restricted thereto.

### Example 1

### Preparation of Malignant Tumor Treatment Agent

After mixing together 3.2 g of dried MUGI KOJI (koji malt) powder, 3.0 g of dried SHIITAKE mushroom (Lentinula edodes) powder, 2.7 g of dried coix seed powder, 2.5 g of dried cacao powder, 1.9 g of dried nutmeg powder, 1.8 g of dried coffee powder, 1.0 g of dried gansun (bamboo shoots) powder, 0.9 g of dried parsley powder, 0.8 g of dried stevia powder, and 0.7 g of dried mint powder, the mixture was pulverized and extracted in 300 g of hot water at approximately 90 degree Celsius for 40 minutes to obtain an extract solution as Example 1.

### Comparative Example 1

### Preparation of Hot Water Extract of Patent Literature 4

After mixing together 3.0 g of dried SHIITAKE mushroom (Lentinula edodes) powder, 3.0 g of dried cacao nib powder, 2.0 g of dried coffee bean powder, and 1.0 g of dried stevia powder, the mixture was pulverized and extracted in 300 g of hot water at approximately 90 degree Celsius for 40 minutes to obtain an extract solution as Comparative Example 1.

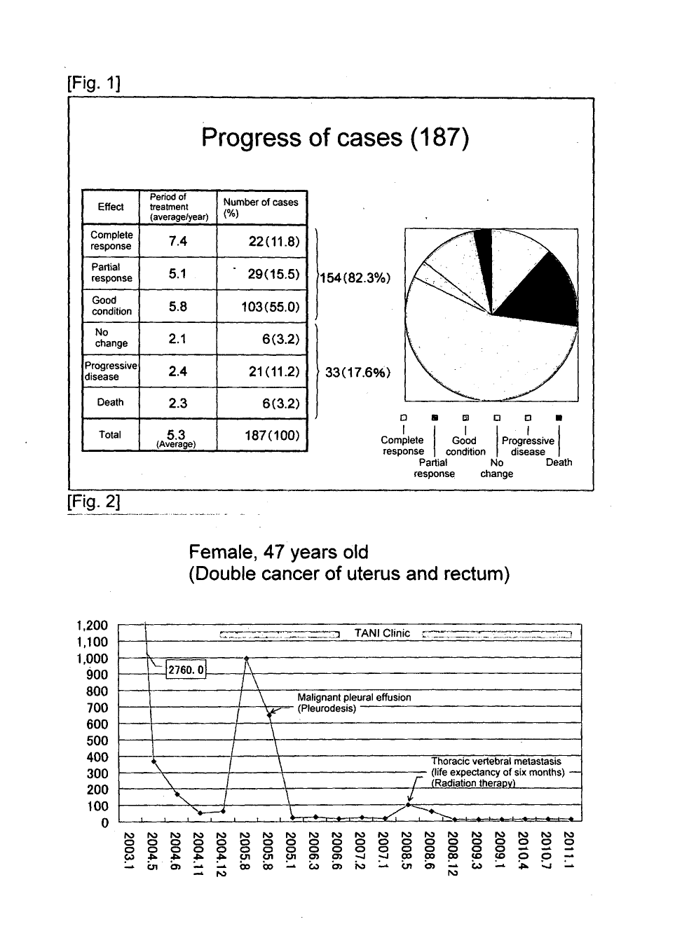

A description will be given of cases to which Example 1 of the present invention was applied. Example 1 was applied to 186 cases in total consisting of 43 breast cancer cases, 21 colon cancer cases, 18 lung cancer cases, 17 stomach cancer cases, 16 prostate cancer cases, 11 uterine cancer cases, 11 malignant lymphoma cases, 11 ovarian cancer cases, 5 liver cancer cases, 5 kidney cancer cases, 5 multiple myeloma cases, 5 leukemia cases, 4 thyroid cancer cases, 4 sarcoma cases, 3 biliary tract cancer cases, 2 pancreatic cancer, and 9 other cancer cases. In the present invention, as shown in FIG. 1, the treatment progress showed that, among the 187 cases, complete response was observed in 22 cases (11.8%); partial response was observed in 29 cases (15.5%); 103 cases (55.0%) showed good condition; 6 cases (3.2%) showed no change; 21 cases (11.2%) had progressive disease; and 6 cases (3.2%) died.

On the other hand, Comparative Example was applied to 106 cases in total, where 19 cases (17.9%) showed tumor disappearance or leukemia remission; 17 cases (16%) had tumor size reduction or significant tumor marker reduction; 57 cases (53%) had surgery for cancer but metastasis to adjacent lymph nodes was observed under microscopy or had improved physical constitution while taking examinations in hospital because of various concerns about reoccurrence; 2 cases (1.8%) had progressive disease; and 11 cases (10.4%) died. In brief, this shows that the execution of Example of the present invention greatly improved the death rate.

According to a more detailed investigation of the 43 breast cancer cases, in procedures and findings at the occurrence of disease, among the 43 cases, 42 cases had surgery; 37 cases had chemotherapy; 18 cases had radiation therapy; 5 cases had positive lymph node resection; and 13 cases had metastasis to other organs (including remote lymph node metastasis), where 3 cases showed complete response; 6 cases showed partial response; 30 cases showed good condition; 2 cases had progressive disease; and 2 cases died (death rate: 4.7%). In addition, regarding the 13 cases with metastasis to other organs (including remote lymph node metastasis), which usually shows high mortality, 3 cases showed complete response; 6 cases showed partial response; 2 cases had progressive disease; and 2 cases died (death rate: 15.4%).

Furthermore, the present invention had a recurrence rate of 4.6%, whereas Comparative Example had a recurrence rate of 30%.

The following is the results of clinical test for confirming the effect of the product of the present invention (Example 1) on treatment of malignant tumor. FIG. 2 shows the progress of tumor marker (CA 125) for 8 years.

PATIENT: Female, 47 years old

DIAGNOSIS: Double cancer of the uterus and rectum

PROGRESS: On November 2003, the patient had double cancer of the uterus (residual adenocarcinoma) and the rectum (tubular adenocarcinoma). Her weight at that time was 55 to 56 kg. She was then hospitalized in surgical hospital. As shown in Table 2, tumor marker CA 125 was 2760 as of January 2003.

On December of the year, 7 courses of chemotherapy were performed.

On May of the next year, 2004, as shown in FIG. 2, the tumor marker CA 125 was approximately 370.

On June of the same year, the patient underwent total uterus extirpation and rectal resection surgery. At that time, a residual metastatic lymph node was found and therefore, a combination treatment of chemotherapy and radiation therapy was started. At that time, the patient's weight was 55.8 kg. The tumor marker CA 125 was approximately 180.

On October of the year, there was observed a clavicular fossa lymph node metastasis. From October 22 to November 12, radiation therapy was performed. Then, her weight decreased to 45 kg. The tumor marker CA 125 was approximately 50.

On December of the year, the patient consulted the applicant. The tumor marker CA 125 was approximately 70.

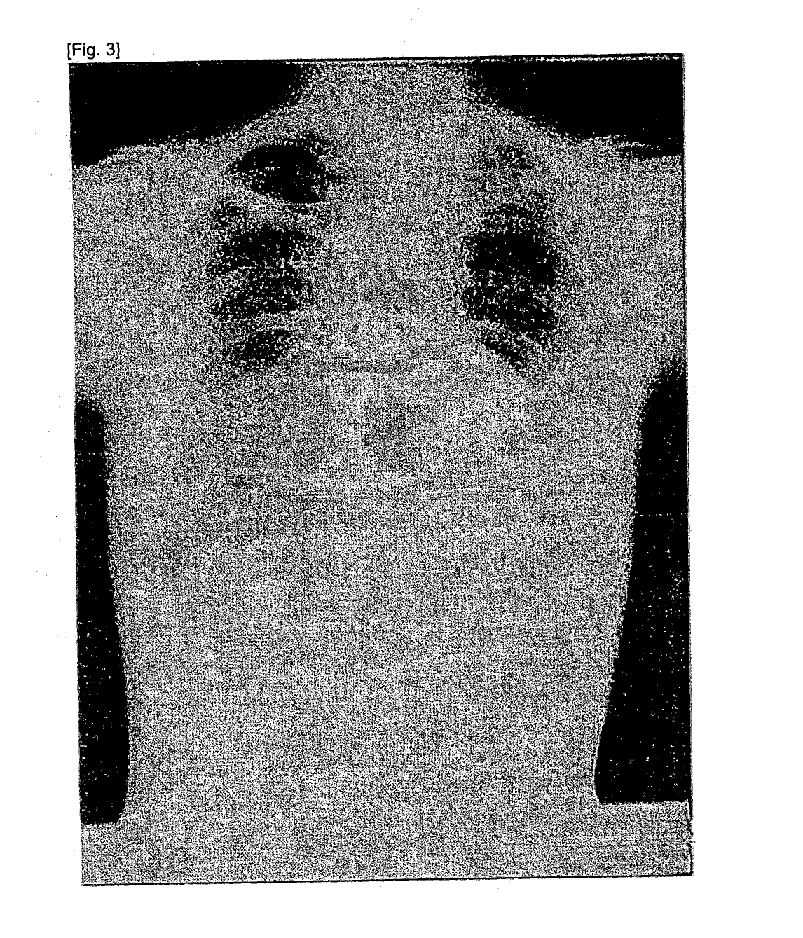

On August 2005, as shown in FIG. 3, there was a shadow in the lungs. The patient had pleurisy and developed cancerous pleural effusion. The tumor marker CA 125 increased to approximately 1000 and decreased to 650. Then, pleurodesis was performed. During the period, the patient came to clinic of the applicant two or three times a week, and from then on, no anticancer drug treatment was provided.

On December of the year, the tumor marker CA 125 decreased to 33.9.

During the period from January of 2006 to January of 2007, the tumor marker CA 125 remained to be 50 or less.

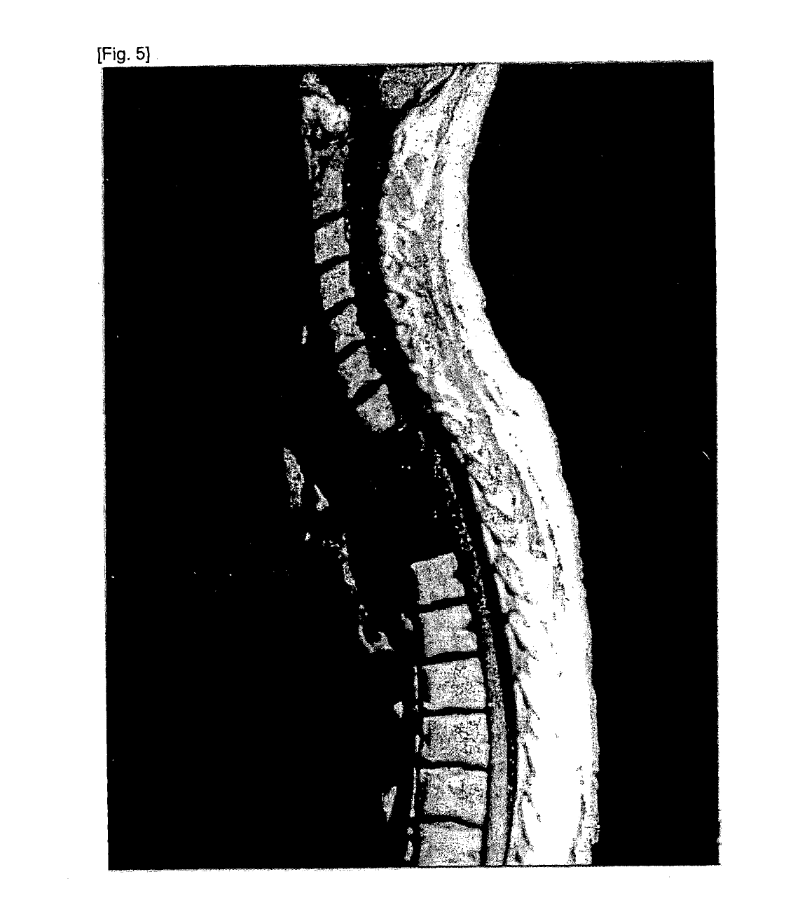

On May 2008, a thoracic vertebral metastasis was found. Paralysis developed in both legs. The tumor marker CA 125 increased to 100. The patient was told that her life expectancy was 6 months. She was recommended to be hospitalized in hospice. FIG. 5 shows an X-ray photograph of the backbone taken at that time. It shows the disappearance of three vertebrae of the backbone due to cancer metastasis. Radiation therapy was performed. Her weight was then 34 kg.

On June of the year, the tumor marker CA 125 decreased to approximately 60.

On November of the year, the patient left hospital and started once-a-week rehabilitation. The paralysis in the legs gradually improved.

On November of the year, the tumor marker CA 125 decreased to approximately 10.

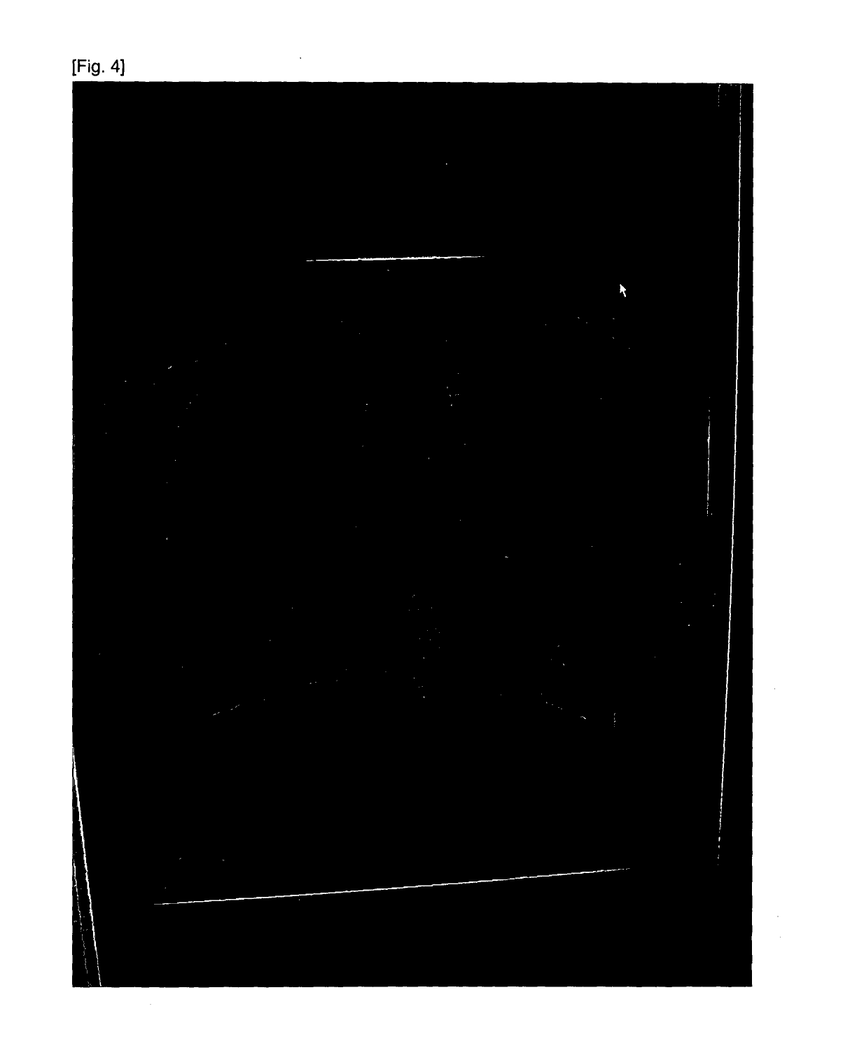

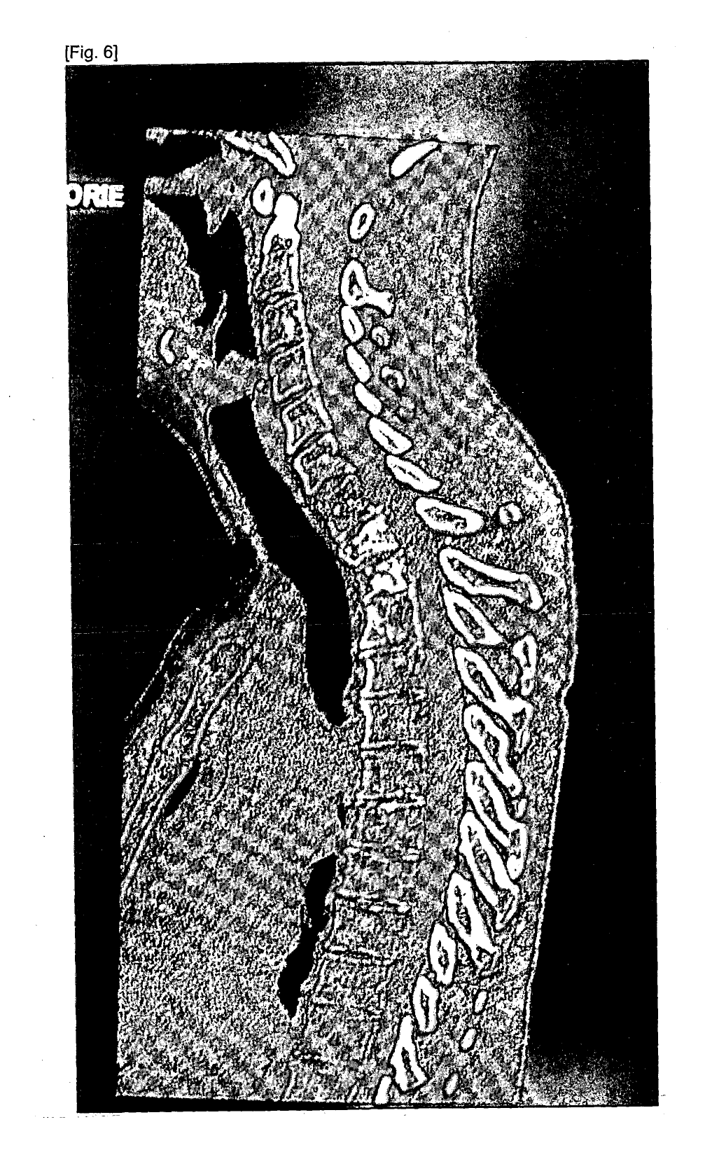

After that, in 2009, metastatic lesions significantly improved. No reoccurrence was observed and the patients' general condition favorably recovered. At that point in time, the shadow in the lungs completely disappeared, as shown in FIG. 4. Additionally, as in FIG. 6, regeneration of the vertebrae was observed.

As of 2011, the patient recovered to enjoy dancing as her hobby. Her weight increased to 62 kg.

1. A malignant tumor treatment agent comprising, as an active ingredient, a mixture obtained by mixing together MUGI KOJI (koji malt), SHIITAKE mushroom (Lentinula edodes), coix seed, cacao, nutmeg, coffee, gansun (bamboo shoots), parsley, stevia, and mint.

2. The malignant tumor treatment agent according to claim 1, comprising, as the active ingredient, an extract obtained by additional hot water extraction of the mixture.

3. The malignant tumor treatment agent according to claim 1, wherein the mixture is in a dry powder state.

4. The malignant tumor treatment agent according to claim 3, wherein a weight ratio in respective dry states of MUGI KOJI (koji malt), SHIITAKE mushroom (Lentinula edodes), coix seed, cacao, nutmeg, coffee, gansun (bamboo shoots), parsley, stevia, and mint is 3.2:3.0:2.7:2.5:1.9:1.8:1.0:0.9:0.8:0.7.

5. Food and drink comprising the malignant tumor treatment agent according to claim 1.

6. The malignant tumor treatment agent according to claim 2, wherein the mixture is in a dry powder state.

7. Food and drink comprising the malignant tumor treatment agent according to claim 2.

8. Food and drink comprising the malignant tumor treatment agent according to claim 3.

9. Food and drink comprising the malignant tumor treatment agent according to claim 4.
